(19) United States
(12) Patent Application Publication
(10) Pub. No.: US 2023/0157754 A1
(43) Pub. Date: May 25, 2023

(54) FASTER RISE TIME PULSE SHAPING OF PLASMA GENERATED PRESSURE WAVES FOR DISRUPTION OF VASCULAR CALCIUM

(71) Applicant: Bolt Medical, Inc., Carlsbad, CA (US)

(72) Inventors: Gerald David Bacher, Carlsbad, CA (US); Christopher A. Cook, Laguna Niguel, CA (US)

(21) Appl. No.: 18/101,178

(22) Filed: Jan. 25, 2023

Related U.S. Application Data
(63) Continuation of application No. 17/190,913, filed on Mar. 3, 2021.
(60) Provisional application No. 63/067,780, filed on Aug. 19, 2020.

(51) Int. Cl. A61B 18/26

(52) U.S. Cl. CPC A61B 18/26; A61B 2018/266; A61B 2018/0022

(57) ABSTRACT

A catheter system (100) includes an inflatable balloon (104), an optical fiber (122), and a laser (124). The optical fiber (122) has a distal end positioned within the inflatable balloon (104). The optical fiber (122) receives an energy pulse (431) to emit light energy in a direction away from the optical fiber (122) to generate a plasma pulse (134) within the inflatable balloon (104). The laser (124) includes a seed source (126) that emits a seed pulse (342) and an amplifier (128) that increases energy of the seed pulse (342) so that the laser (124) generates the energy pulse (431) that is received by the optical fiber (122), the energy pulse (431) having a waveform with a duration T, a minimum power PO, a peak power PP, and a time from PO to PP equal to TP.

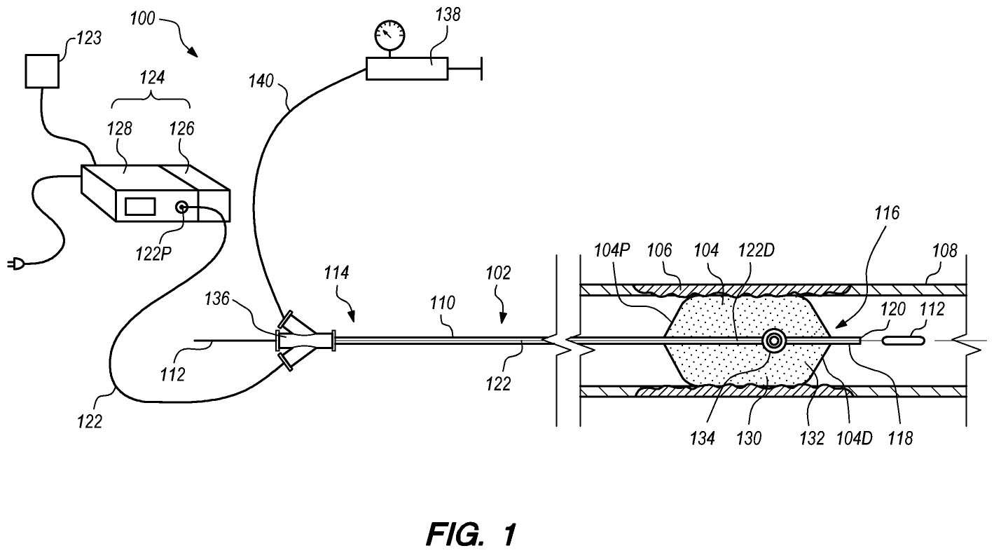

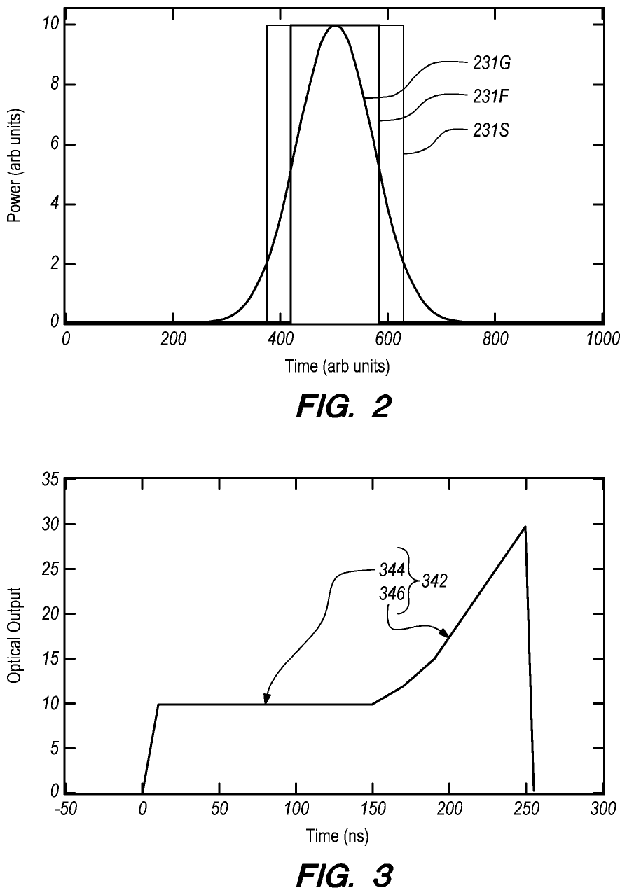

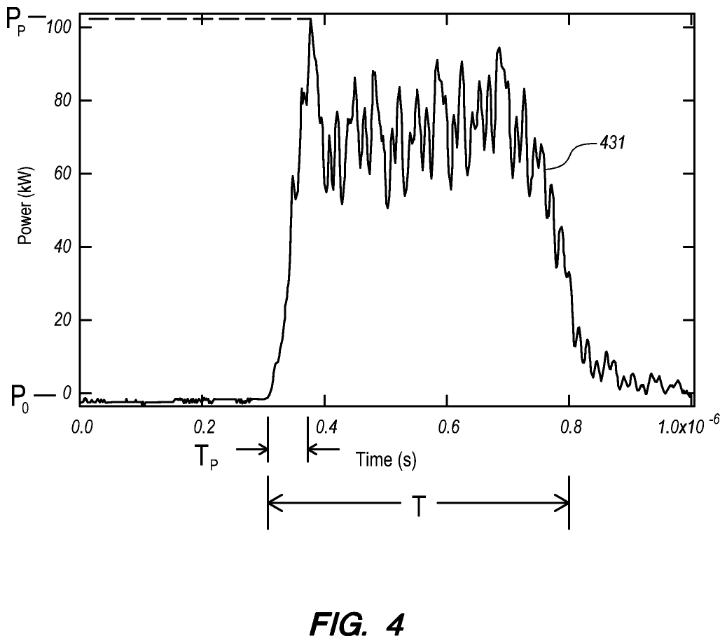

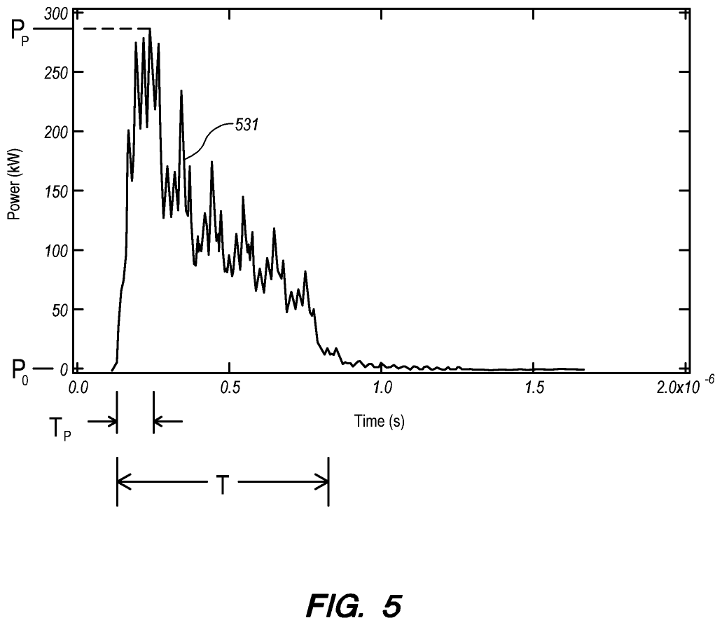

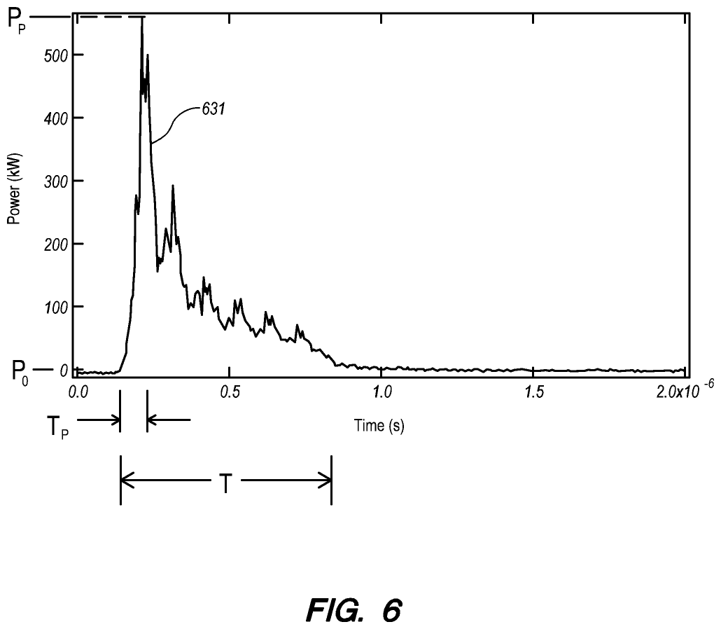

The present invention is directed toward a catheter system for treating a treatment site within or adjacent to a vessel wall or a heart valve. In various embodiments, the catheter system includes an inflatable balloon, an optical fiber, and a laser. The optical fiber has a fiber distal end positioned within the inflatable balloon. The optical fiber is configured to receive an energy pulse so that the optical fiber emits light energy in a direction away from the optical fiber to generate a plasma pulse within the inflatable balloon. The laser includes (i) a seed source that is configured to emit a seed pulse, and (ii) an amplifier that is configured to increase energy of the seed pulse so that the laser generates the energy pulse that is received by the optical fiber, the energy pulse having a waveform with a duration T, a minimum power P₀, a peak power P_{P}, and a time from P₀ to P_{P} equal to T_{P}, wherein T_{P} is not greater than 40% of T, wherein P_{P} is within the range of greater than 50 kW and less than 1000 kW.

In certain embodiments, wherein T_{P} is not greater than 30% of T.

In some embodiments, wherein T_{P} is not greater than 25% of T.

In various embodiments, wherein T_{P} is not greater than 20% of T.

In certain embodiments, wherein T_{P} is not greater than 10% of T.

In some embodiments, wherein T_{P} is not greater than 5% of T.

In various embodiments, wherein T is within the range of greater than 50 ns and less than 3 μs.

In certain embodiments, wherein T is within the range of greater than 100 ns and less than 2 μs.

In some embodiments, wherein T is within the range of greater than 200 ns and less than 1 μs.

In various embodiments, wherein T is within the range of greater than 300 ns and less than 800 ns.

The present invention is also directed toward a catheter system for treating a treatment site within or adjacent to a vessel wall or a heart valve. In various embodiments, the catheter system includes an inflatable balloon, an optical fiber, and a laser. The optical fiber has a fiber distal end positioned within the inflatable balloon. The optical fiber is configured to receive an energy pulse so that the optical fiber emits light energy in a direction away from the optical fiber to generate a plasma pulse within the inflatable balloon. The laser includes (i) a seed source that is configured to emit a seed pulse, and (ii) an amplifier that is configured to increase energy of the seed pulse so that the laser generates the energy pulse that is received by the optical fiber, the energy pulse having a waveform with a duration T, a minimum power P₀, a peak power P_{P}, and a time from P₀ to P_{P} equal to T_{P}, wherein T_{P} is not greater than 40% of T, wherein a ratio in kW to ns of P_{P} to T_{P} is greater than 1:5.

In certain embodiments, T is within the range of greater than 50 ns and less than 3 μs.

In some embodiments, T is within the range of greater than 100 ns and less than 2 μs.

In various embodiments, T is within the range of greater than 200 ns and less than 1 μs.

In certain embodiments, T is within the range of greater than 300 ns and less than 800 ns.

In some embodiments, T is within the range of greater than 400 ns and less than 600 ns.

In various embodiments, T_{P} is within the range of greater than 2.5 ns and less than 1 μs.

In certain embodiments, T_{P} is within the range of greater than 5 ns and less than 800 ns.

The present invention is further directed toward a catheter system for treating a treatment site within or adjacent to a vessel wall or a heart valve. In various embodiments, the catheter system includes an inflatable balloon, an optical fiber, and a laser. The optical fiber has a fiber distal end positioned within the inflatable balloon. The optical fiber is configured to receive an energy pulse so that the optical fiber emits light energy in a direction away from the optical fiber to generate a plasma pulse within the inflatable balloon. The laser includes (i) a seed source that is configured to emit a seed pulse, and (ii) an amplifier that is configured to increase energy of the seed pulse so that the laser generates the energy pulse that is received by the optical fiber, the energy pulse having a waveform that approximates a square wave, the waveform having a duration T, a minimum power P₀, a peak power P_{P}, and a time from P₀ to P_{P} equal to T_{P}, wherein T_{P} is greater than 40% of T.

The present invention is still further directed toward a catheter system for treating a treatment site within or adjacent to a vessel wall or a heart valve. In various embodiments, the catheter system includes an inflatable balloon, an optical fiber, and a laser. The optical fiber has a fiber distal end positioned within the inflatable balloon. The optical fiber is configured to receive an energy pulse so that the optical fiber emits light energy in a direction away from the optical fiber to generate a plasma pulse within the inflatable balloon. The laser includes (i) a seed source that is configured to emit a seed pulse, and (ii) an amplifier that is configured to increase energy of the seed pulse so that the laser generates the energy pulse that is received by the optical fiber, the energy pulse having a waveform that approximates a triangular wave, the waveform having a duration T, a minimum power P₀, a peak power P_{P}, and a time from P₀ to P_{P} equal to T_{P}, wherein T_{P} is greater than 40% of T, wherein T_{P} is greater than 60% of T.

This summary is an overview of some of the teachings of the present Application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

1. A catheter system for treating a treatment site within or adjacent to a vessel wall or a heart valve, the catheter system comprising:
an inflatable balloon;
an optical fiber having a fiber distal end positioned within the inflatable balloon, the optical fiber being configured to receive an energy pulse so that the optical fiber emits light energy in a direction away from the optical fiber to generate a plasma pulse within the inflatable balloon; and
a laser including (i) a seed source that is configured to emit a seed pulse, and (ii) an amplifier that is configured to increase energy of the seed pulse so that the laser generates the energy pulse that is received by the optical fiber, the energy pulse having a waveform with a duration T, a minimum power P₀, a peak power P_{P}, and a time from P₀ to P_{P} equal to T_{P}, wherein T_{P} is not greater than 40% of T, wherein P_{P} is within the range of greater than 50 kW and less than 1000 kW.

2. The catheter system of claim 1 wherein T_{P} is not greater than 30% of T.

3. The catheter system of claim 1 wherein T_{P} is not greater than 25% of T.

4. The catheter system of claim 1 wherein T_{P} is not greater than 20% of T.

5. The catheter system of claim 1 wherein T_{P} is not greater than 10% of T.

6. The catheter system of claim 1 wherein T_{P} is not greater than 5% of T.

7. The catheter system of claim 1 wherein T is within the range of greater than 50 ns and less than 3 μs.

8. The catheter system of claim 1 wherein T is within the range of greater than 100 ns and less than 2 μs.

9. The catheter system of claim 1 wherein T is within the range of greater than 200 ns and less than 1 μs.

10. The catheter system of claim 1 wherein T is within the range of greater than 300 ns and less than 800 ns.

11. A catheter system for treating a treatment site within or adjacent to a vessel wall or a heart valve, the catheter system comprising:
an inflatable balloon;
an optical fiber having a fiber distal end positioned within the inflatable balloon, the optical fiber being configured to receive an energy pulse so that the optical fiber emits light energy in a direction away from the optical fiber to generate a plasma pulse within the inflatable balloon; and
a laser including (i) a seed source that is configured to emit a seed pulse, and (ii) an amplifier that is configured to increase energy of the seed pulse so that the laser generates the energy pulse that is received by the optical fiber, the energy pulse having a waveform with a duration T, a minimum power P₀, a peak power P_{P}, and a time from P₀ to P_{P} equal to T_{P}, wherein T_{P} is not greater than 40% of T, wherein a ratio in kW to ns of P_{P} to T_{P} is greater than 1:5.

12. The catheter system of claim 11 wherein T is within the range of greater than 50 ns and less than 3 μs.

13. The catheter system of claim 11 wherein T is within the range of greater than 100 ns and less than 2 μs.

14. The catheter system of claim 11 wherein T is within the range of greater than 200 ns and less than 1 μs.

15. The catheter system of claim 11 wherein T is within the range of greater than 300 ns and less than 800 ns.

16. The catheter system of claim 11 wherein T is within the range of greater than 400 ns and less than 600 ns.

17. The catheter system of claim 12 wherein T_{P} is within the range of greater than 2.5 ns and less than 1 μs.

18. The catheter system of claim 12 wherein T_{P} is within the range of greater than 5 ns and less than 800 ns.

19. A catheter system for treating a treatment site within or adjacent to a vessel wall or a heart valve, the catheter system comprising:
an inflatable balloon;
an optical fiber having a fiber distal end positioned within the inflatable balloon, the optical fiber being configured to receive an energy pulse so that the optical fiber emits light energy in a direction away from the optical fiber to generate a plasma pulse within the inflatable balloon; and
a laser including (i) a seed source that is configured to emit a seed pulse, and (ii) an amplifier that is configured to increase energy of the seed pulse so that the laser generates the energy pulse that is received by the optical fiber, the energy pulse having a waveform that approximates a square wave, the waveform having a duration T, a minimum power P₀, a peak power P_{P}, and a time from P₀ to P_{P} equal to T_{P}, wherein T_{P} is greater than 40% of T.

20. A catheter system for treating a treatment site within or adjacent to a vessel wall or a heart valve, the catheter system comprising:
an inflatable balloon;
an optical fiber having a fiber distal end positioned within the inflatable balloon, the optical fiber being configured to receive an energy pulse so that the optical fiber emits light energy in a direction away from the optical fiber to generate a plasma pulse within the inflatable balloon; and
a laser including (i) a seed source that is configured to emit a seed pulse, and (ii) an amplifier that is configured to increase energy of the seed pulse so that the laser generates the energy pulse that is received by the optical fiber, the energy pulse having a waveform that approximates a triangular wave, the waveform having a duration T, a minimum power P₀, a peak power P_{P}, and a time from P₀ to P_{P} equal to T_{P}, wherein T_{P} is greater than 40% of T, wherein T_{P} is greater than 60% of T.
